(19) United States
(12) Patent Application Publication
(10) Pub. No.: US 2026/0358309 A1
(43) Pub. Date: Nov. 12, 2026

(54) METHOD FOR SIMULTANEOUSLY MEASURING CONTENT OF ASPHALTENE AND BENZENE INSOLUBLE SUBSTANCE

(71) Applicant: PetroChina Company Limited, Beijing (CN)

(72) Inventors: Dejun Shi, Beijing (CN); Shengbao He, Beijing (CN); Fei Chen, Beijing (CN); Hongyue Yu, Beijing (CN); Chunyan Wang, Beijing (CN); Mingchen Huo, Beijing (CN); Da Huo, Beijing (CN); Yawen Bo, Beijing (CN); Shang Zhang, Beijing (CN); Kebin Chi, Beijing (CN)

(21) Appl. No.: 19/140,150

(22) PCT Filed: Aug. 23, 2023
(86) PCT No.: PCT/CN2023/114468

§ 371 Date: Jun. 17, 2025

(30) Foreign Application Priority Data
Dec. 27, 2022 (CN) 202211680493.9

(51) Int. Cl. G01N 30/88; G01N 3/50; G01N 30/06; G01N 30/16; G01N 30/34; G01N 33/28

(52) U.S. Cl. CPC G01N 30/88; G01N 3/50; G01N 30/06; G01N 30/16; G01N 30/34; G01N 33/2823; G01N 2030/167; G01N 2030/884

(57) ABSTRACT

Provided in the present invention is a method for simultaneously measuring the content of asphaltene and a benzene insoluble substance. The method comprises: mixing a sample to be measured and a diluent under heating conditions until uniform, and placing the obtained mixture in a sample injection pipe; activating a dried separation column by means of a diluent; adding all the mixture in the sample injection pipe to the upper portion of the separation column; flushing the separation column with a diluent; flushing the separation column with a benzene series eluent and collecting an effluent by using a dried collection bottle, and stopping flushing until the effluent is colorless and respectively calculating the content of the asphaltene and the benzene insoluble substance in the oil slurry sample to be measured. The method has the advantages of simplicity, reliability, high repeatability, high analysis efficiency, a low solvent amount, etc.

The present application claims priority to Chinese patent application No. 202211680493.9, entitled “METHOD FOR SIMULTANEOUSLY MEASURING CONTENTS OF ASPHALTENES AND BENZENE-BASED INSOLUBLE SUBSTANCES BY SOLID PHASE EXTRACTION SEPARATION”, filed with the Chinese Patent Office on Dec. 27, 2022, the entire contents of which are incorporated herein by reference.

### FIELD OF TECHNOLOGY

The disclosure relates to a method for simultaneously measuring the contents of asphaltenes and benzene-based insoluble substances, and belongs to the technical field of chemical analysis.

### BACKGROUND OF ART

With the decreasing of conventional petroleum resources and the increasing trend of heavy and deteriorated crude oil, high-value processing processes of heavy oil has attracted more and more attention of researchers. Asphaltenes refer to components in petroleum or petroleum fractions that are insoluble in alkane solvents (typically, n-heptane or n-pentane as a precipitant) but soluble in aromatic solvents such as benzene or toluene. The content of asphaltenes is an important indicator for evaluating the difficulty of processing crude oil and heavy oil, wherein the coking tendency of the oil and the amount of coke in the coking process has a correlation with the content of asphaltenes. Toluene insoluble substances are components that are more polar than asphaltenes and are generally considered as coking precursors. The process of heavy oil lightening and high-value processing is generally carried out at high temperature and high pressure, during which a large amount of coking and caking occurs, and one of the factors affecting the coking and caking is the presence of toluene insoluble substances. The mass fraction of toluene insoluble substances would directly affect the conversion rate of heavy oil lightening. Therefore, the determination of the contents of asphaltenes and toluene insoluble substances is of great concern in the processing of heavy oil and crude oil.

The current method for measuring benzene-based insoluble substances in China is mainly GB/T 2559.7-2005 “Analysis of lignite wax-Method for measuring benzene insolubles in lignite wax”, wherein the method for measuring toluene insoluble substances is GB/T 2292-2018 “Determination of toluene insoluble content for coking products”, GB/T 8926-2012 “Standard test method for insolubles in used lubricating oils”, and the like. Generally, the sample is dissolved directly with benzene or toluene, and benzene or toluene insoluble substances are separated by liquid-liquid extraction or centrifugation. However, the above methods can only measure the content of benzene-based insoluble substances, and a large amount of benzene or toluene solvent is used, which is time-consuming and complicated in operation. Current methods for measuring asphaltenes includes SH/T 0509-2010 “Test method for separation of asphalt into four fractions”, YB/T 5178-2016 “Feedstock oil for carbon black-Determination of asphaltine content-Precipitation of n-heptane”, SY/T 7550-2012 “Determination of wax, resins and asphaltenes in Crude Oil”, and SN/T 3118-2012 “Determination of asphaltenes in residual oil-Claviform thin-layer chromatography”. Among these, the first three methods generally use n-heptane to precipitate asphaltenes, and obtain the asphaltenes by removing the solubles in the precipitate through reflux with n-heptane after filtration, followed by recovering and dissolving the precipitate with toluene, in which the amount of reagent is large, the experimental time is long, and the environmental pollution is serious. Although the last method uses a small amount of solvent and is simple and fast, there are problems such as low separation efficiency, different response coefficients of different components and oil species, and poor accuracy and reproducibility. The above four analysis methods cannot obtain the contents of asphaltenes and toluene insoluble substances simultaneously.

Some prior art situations related to the disclosure will be briefly introduced below, and the disadvantages of the prior art will be analyzed and illustrated.

Prior art document 1: Method for continuous determination of toluene insoluble and asphaltenes in heavy oil [J]. Petroleum Processing and Petrochemicals, 2018, 49 (02): 104-106. discloses a method for simultaneously measuring asphaltenes and toluene insoluble substances, in which the test methods in GB/T 2992-1997 and SY/T 7550-2012 are combined, and the two experiments are combined together; a sample is dissolved and extracted with n-heptane in a filter paper cylinder by using a single device; the soluble substances are discarded, and then the n-heptane insoluble substances in the filter paper cylinder are dissolved and extracted with toluene; the solvent is removed from the obtained toluene solution, which is allowed to have a constant weight and weighted to calculate the content of asphaltenes; the insoluble substances in the filter paper cylinder are allowed to have a constant weight and calculated for the content of toluene insoluble substances. Although this method could give the contents of asphaltenes and toluene insoluble substances simultaneously, the steps are complicated, and the determination of one sample takes up to 18 h. Moreover, the use of a filter paper cylinder as a separation carrier has mass loss during the elution process and results in higher experimental results.

Prior art document 2: Chinese patent CN 106947515A discloses a method for solid phase extraction separation of different types of compounds in crude oil or heavy oil, comprising: adding a crude oil or heavy oil sample into a stationary phase, washing the stationary phase with C5-C7 saturated hydrocarbon, extracting saturated hydrocarbon components in the sample, washing the stationary phase with chlorinated alkane, extracting aromatic hydrocarbon components in the sample, and washing the stationary phase with dichloromethane-ethanol mixture or ethanol to obtain resins, wherein the stationary phase comprises a water-soluble salt of a Group IB element or a Group iron element supported in alumina, wherein the content of the water-soluble salt of the Group IB element or the iron-group element is 2.0-10.0 mass % based on alumina. This method can effectively separate saturated hydrocarbons and aromatic hydrocarbons in crude oil or heavy oil samples, and each component obtained after separation can be directly used for instrument analysis, thereby obtaining the group compositions of the analysis samples. However, the amount of the sample treated by this method is only 0.4 g, and when the amount of the sample is increased, there is a problem that insoluble substances in the oil sample block the filter holes of the sieve plate, such that the analysis cannot be continued. Moreover, this method can only obtain the mass of soluble samples such as saturated hydrocarbons, aromatic hydrocarbons, resins and the like, and cannot directly obtain the content of insoluble substances. Furthermore, this method generally employs polar fillers, with irreversible adsorption for some strongly polar substances.

Therefore, it has become an urgent technical problem to be solved in this field to provide a novel method for simultaneously measuring the contents of asphaltenes and benzene-based insoluble substances.

### SUMMARY OF INVENTION

In order to solve the above-mentioned disadvantages and shortcomings, one object of the disclosure is to provide a method for simultaneously measuring the contents of asphaltenes and benzene-based insoluble substances. The method of this disclosure simultaneously separates asphaltenes and benzene-based insoluble substances, and can be used for the separation and measurement of asphaltenes and benzene-based insoluble substances in products such as coal chemical products and petrochemical products.

In order to achieve the above object, in one aspect, the disclosure provides a method for simultaneously measuring the contents of asphaltenes and benzene-based insoluble substances, comprising steps of:
(1) mixing a sample to be measured with a diluent under heating conditions until uniform and placing the obtained mixture in a sample injection tube;
(2) activating a dried separation column with the diluent;
(3) adding all the mixture in the sample injection tube to the top of the separation column;
(4) rinsing the separation column with the diluent to obtain alkane insoluble substances; then rinsing the separation column with a benzene-based eluent, and collecting an effluent with a dried collection bottle and stopping rinsing until the effluent is colorless, whereby asphaltenes are in the collection bottle, and benzene-based insoluble substances remain on the separation column;
(5) drying the collection bottle and the separation column in step (4); and
(6) calculating the content of each of the asphaltenes and the benzene-based insoluble substances according to the mass of the sample to be measured, the mass difference of the collection bottle and the mass difference of the separation column.

In a specific embodiment of the method of the disclosure, the method further comprises, between step (3) and step (4), washing the sample injection tube with the diluent and adding all the resultant washing liquid to the top of the separation column.

In a specific embodiment of the method of the disclosure, the diluent comprises one or more of n-pentane, n-hexane, n-heptane, petroleum ether and the like.

In a specific embodiment of the method of the disclosure, the sample to be measured comprises, but is not limited to, crude oil, heavy oil, tar or asphalt products in coal chemical industry or petrochemical industry.

In a specific embodiment of the method of the disclosure, the amount of the sample to be measured is 0.1-100 g.

In a specific embodiment of the method of the disclosure, the volume of the separation column is 6-150 mL, preferably 20-40 mL.

In a specific embodiment of the method of the disclosure, a filter material used in the separation column has a pore size of 0.1-200 μm.

In a specific embodiment of the method of the disclosure, the filter material comprises a neutral filter paper, a filter cloth, a glass sand core, a polymeric material or the like, preferably a polymeric material. In some embodiments of the disclosure, the polymeric material may be, for example, polyethylene, a Teflon material or the like.

In a specific embodiment of the method of the disclosure, the filling height of the filler used in the separation column is 5%-80% of the total height of the separation column, and the particle size of the filler is in the range of 20-300 mesh.

In a specific embodiment of the method of the disclosure, the filler used in the separation column is a weakly polar material and/or an inert material, etc.

In a specific embodiment of the method of the disclosure, the filler is quartz sand or a mixture of quartz sand and at least one selected from the group consisting of calcium sulfate, alumina, silica gel, diatomaceous earth, clay, polymer, barite powder, and the like. In some embodiments of the disclosure, the polymer may be, for example, epoxy resin, polypropylene, polymethyl methacrylate, or the like.

In a specific embodiment of the method of the disclosure, the content of quartz sand in the filler is 70%-100%, based on 100% of the total weight of the filler.

In a specific embodiment of the method of the disclosure, in step (1), the mass ratio of the sample to be measured to the diluent is 1:1 to 1:50; in step (2), the amount of the diluent is 3-20 mL.

In a specific embodiment of the method of the disclosure, the amount of the diluent used for washing the sample injection tube is 5-50 mL.

In a specific embodiment of the method of the disclosure, in step (4), the amount of the diluent is 1-10 times the amount of the diluent in step (2), and the amount of the benzene-based eluent is 1-10 times the amount of the diluent in step (2).

In a specific embodiment of the method of the disclosure, the benzene-based eluent comprises benzene and/or toluene, etc.

In a specific embodiment of the method of the disclosure, in step (2), step (4) and step (5), the temperature for drying is 100-150 °C.

In a specific embodiment of the method of the disclosure, in step (1), the temperature for heating is 50-100 °C. That is, the temperature of the mixture obtained after uniformly mixing the sample to be measured and the diluent in step (1) is 20-100 °C.

In a specific embodiment of the method of the disclosure, in step (2), the temperature of the diluent is 20-100 °C.

In a specific embodiment of the method of the disclosure, in step (4), the temperature of the diluent and the benzene-based eluent is 20-100 °C.

In a specific embodiment of the method of the disclosure, the temperature of the diluent used for washing the sample injection tube is 20-100 °C.

In a specific embodiment of the method of the disclosure, in step (6), the content of each of the asphaltenes and the benzene-based insoluble substances in the sample to be measured is calculated from the mass of the sample to be measured, the mass difference of the collection bottle and the mass difference of the separation column, according to the following Equation 1) and Equation 2):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

in Equation 1) and Equation 2), W1 is the mass percentage (%) of the asphaltenes in the sample to be measured;
W2 is the mass percentage (%) of the benzene-based insoluble substances in the sample to be measured;
m₁ is the mass (g) of the dried collection bottle used in step (4);
m₂ is the mass (g) of the dried separation column in step (2);
m₃ is the mass (g) of the sample to be measured;
m₄ is the mass (g) of the collection bottle after drying in step (5); and
m₅ is the mass (g) of the separation column after drying in step (5).

The method for simultaneously measuring the contents of asphaltenes and benzene-based insoluble substances by solid phase extraction separation provided by the disclosure can be applied to the determination of asphaltenes and benzene-based insoluble substances in samples of coal chemical industry, petrochemical industry, or the like. The method is simple and reliable, has high reproducibility, high analysis efficiency and low solvent consumption, and can realize the automation of the analysis process by means of instruments. It solves the problems (such as instrument error introduced by the separation consumables, low extraction efficiency/low separation efficiency, complicated steps, long separation time/long time consumption, and large solvent consumption) of the traditional methods for separating insoluble substances based on liquid-liquid extraction method or centrifugal separation, and it has high promotion value and application potential. In addition, the method of the disclosure can also be implemented separately to separate alkane insoluble substances and benzene-based insoluble substances in samples of coal chemical industry, petrochemical industry, or the like.

### DETAILED DESCRIPTION OF PREFERRED EMBODIMENTS

It should be noted that the term “comprise” and any variations thereof in the description and claims of the disclosure are intended to cover non-exclusive inclusion, for example, a process, method, system, product or apparatus comprising a series of steps or units is not necessarily limited to those steps or units explicitly listed, but may include other steps or units that are not explicitly listed or are inherent to such process, method, product or apparatus.

In the disclosure, the terms referring to an orientational or positional relationship, such as “above”, “below”, “inside”, “outside” and “in”, are used primarily to better describe the disclosure and examples thereof, and are not intended to define that the indicated device, element, or component must have a particular orientation or be constructed and operated in a particular orientation.

In addition, some of the above terms may be used to denote other meanings in addition to orientation or location relationships. For example, the term “on” may also be used in some instances to indicate a dependency or connection relationship. For those of ordinary skill in the art, the specific meaning of these terms in the disclosure can be understood according to specific circumstances.

The range disclosed in the disclosure is given in the form of a lower limit and an upper limit. There can be one or more lower limits, and one or more upper limits, respectively. The given range is defined by selecting a lower limit and an upper limit. The selected lower limit and upper limit define the boundary of a particular range. All ranges defined as such are combinable, that is, any lower limit can be combined with any upper limit to form a range. For example, where ranges of 60-120 and 80-110 are listed for a particular parameter, it is understood that the ranges of 60-110 and 80-120 are also contemplated. Furthermore, if the listed minimum values of the range are 1 and 2, and the listed maximum values of the range are 3, 4, and 5, then the following ranges are all contemplated: 1-3, 1-4, 1-5, 2-3, 2-4, and 2-5.

In the disclosure, the numerical range “a-b” represents an abbreviation for any combination of real numbers between a and b, wherein a and b are both real numbers, unless specified otherwise. For example, the numerical range “0-5” means that all real numbers among “0-5” have been listed in the disclosure, and “0-5” is just an abbreviation of these numerical combinations.

In the disclosure, all embodiments and preferable embodiments mentioned in the disclosure can be combined with each other to form a new technical solution, unless specified otherwise.

In the disclosure, all technical features and preferable features mentioned in the disclosure can be combined with each other to form a new technical solution, unless specified otherwise.

In order to make the object, technical solutions and advantages of the disclosure clearer, the disclosure will be further described in detail below in conjunction with examples. The examples described below are part of examples of the disclosure, not exhaustive, and are only used to illustrate the disclosure, and should not be considered as limiting the scope of the disclosure. All other examples obtained by those skilled in the art on the basis of examples in the disclosure without inventive work are within the protection scope of the present disclosure. If no specific conditions are specified in the examples, it is carried out according to conventional conditions or conditions recommended by the manufacturer. If the manufacturer is not specified for the used reagents or instruments, they are all conventional products that are commercially available.

### Example 1

This example provides a method for simultaneously measuring the contents of n-heptane asphaltenes and toluene insoluble substances in a petrochemical asphalt sample by solid phase extraction separation, which comprises the following specific steps.

### Step 1. Preparation

A clean sample injection tube, a collection bottle and a 20 mL separation column filled with a filler were dried at 100 °C for 5 h and then placed in a desiccator, and cooled to room temperature for use.

Herein, the filler used in the separation column was a mixture of 1% alumina, 1% silica gel, 1% diatomaceous earth, 1% clay, 1% epoxy resin, 3% polypropylene, 8% polymethyl methacrylate, 1% barite powder, and 83% quartz sand, based on 100% of the total weight of the filler. The particle size of the filler was 100-200 mesh, and the filling height was 80% of the height of the separation column. A neutral filter paper having a pore size of 0.1 μm was used as the lower filter material, and a glass sand core having a pore size of 200 μm was used as the upper filter material.

Step 2. Sample Preparation 0.1258 g of a petrochemical asphalt sample was diluted with n-heptane at a mass ratio of 1:50, ultrasonically oscillated at 60 °C, and uniformly mixed, and the obtained mixture was placed in the sample injection tube.

The masses of the collection bottle and the separation column were recorded.

### Step 3. Activation

20 mL of n-heptane at 60 °C was added to the top of the separation column to activate the separation column.

### Step 4. Sample Injection

All the sample at 60 °C in the sample injection tube was added to the top of the separation column.

### Step 5. Washing

The sample injection tube was washed twice with 10 mL of n-heptane at 60 °C, and all the washing liquid was added to the top of the separation column, and the separation column was rinsed with the washing liquid.

### Step 6. Rinsing

The separation column was rinsed with 50 mL of n-heptane at 60 °C to obtain n-heptane insoluble substances.

A dried collection bottle was replaced, and the separation column was rinsed with 50 mL of toluene at 70 °C, whereby n-heptane asphaltenes entered the collection bottle, and toluene insoluble substances remained on the separation column.

### Step 7. Drying

The n-heptane in the collection bottle was evaporated. The evaporated collection bottle and the separation column were placed in an oven at 100 °C, dried for 2 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the n-heptane asphaltenes and the toluene insoluble substances was calculated from the mass of the petrochemical asphalt sample, the mass difference of the collection bottle and the mass difference of the separation column, according to the following Equation 1) and Equation 2):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

in Equation 1) and Equation 2), W1 is the mass percentage (%) of asphaltenes in the sample to be measured;
W2 is the mass percentage (%) of benzene-based insoluble substances in the sample to be measured;
m₁ is the mass of the dried collection bottle used in step 6;
m₂ is the mass (g) of the dried separation column (filled with the filler) in step 1;
m₃ is the mass (g) of the sample to be measured;
m₄ is the mass (g) of the collection bottle after drying in step 7;
m₅ is the mass (g) of the separation column after drying in step 7.

In the example, at the stage of sample preparation, the mass of the petrochemical asphalt sample, that is, m₃ was 0.1258 g; the mass of the collection bottle, that is, m₁ was 16.8534 g; the mass of the separation column, that is, m₂ was 21.0245 g; at the stage of measurement of insoluble substances, the mass of the collection bottle, that is, m₄ was 16.8779 g; and the mass of the separation column, that is, m₅ was 21.0385 g. Thus, in the petrochemical asphalt sample, the mass of the n-heptane asphaltenes was 0.0245 g, and the mass of the toluene insoluble substances was 0.0140 g; the mass content of the n-heptane asphaltenes was 19.47%, and the mass content of the toluene insoluble substances was 11.13% in the petrochemical asphalt sample.

The measurement of the above sample was repeated once, and the results showed that the mass content of the n-heptane asphaltenes was 19.40%, and the mass content of the toluene insoluble substances was 11.18% in the sample.

It can be seen that the insoluble substance separation process in the method provided by the example of the disclosure is simple and fast, and the whole separation process of the n-heptane asphaltenes and the toluene insoluble substances takes only 30 min, which greatly improves the analysis speed. The reproducibility of the obtained mass contents of the n-heptane asphaltenes and the toluene insoluble substances is high with deviations of only 0.04% and 0.05%, respectively.

### Example 2

This example provides a method for simultaneously measuring the contents of n-pentane asphaltenes and toluene insoluble substances in a catalytic cracking pilot product of wax oil by solid phase extraction separation, which comprises the following specific steps.

### Step 1. Preparation

A clean sample injection tube, a collection bottle and a 150 mL separation column filled with a filler were dried at 110 °C for 2 h and then placed in a desiccator, and cooled to room temperature for use.

Herein, the filler used in the separation column was a mixture of 5% diatomaceous earth and 95% quartz sand, based on 100% of the total weight of the filler. The particle size of the filler was 40-60 mesh, and the filling height was 5% of the height of the separation column. A filter cloth having a pore size of 10 μm was used as the lower filter material, and polyethylene having a pore size of 200 μm was used as the upper filter material.

### Step 2. Sample Preparation

100.5462 g of the catalytic cracking pilot product of wax oil was diluted with n-pentane at a mass ratio of 1:1, ultrasonically oscillated at 50 °C and uniformly mixed, and the resulting mixture was placed in the sample injection tube.

The masses of the collection bottle and the separation column were recorded.

### Step 3. Activation

10 mL of n-pentane at 20 °C was added to the top of the separation column to activate the separation column.

### Step 4. Sample Injection

All the sample at 50 °C in the sample injection tube was added to the top of the separation column.

### Step 5. Washing

The sample injection tube was washed once with 5 mL of n-pentane at 20 °C, and all the washing liquid was added to the top of the separation column, and the separation column was rinsed with the washing liquid.

### Step 6. Rinsing

The separation column was rinsed with 10 mL of n-pentane at 20 °C to obtain n-pentane insoluble substances.

A dried collection bottle was replaced, and the separation column was rinsed with 10 mL of toluene at 100 °C, whereby n-pentane asphaltenes entered the collection bottle, and toluene insoluble substances remained on the separation column.

### Step 7. Drying

The n-pentane in the collection bottle was evaporated. The evaporated collection bottle and the separation column were placed in an oven at 110 °C, dried for 2 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the n-pentane asphaltenes and the toluene insoluble substances was calculated from the mass of the catalytic cracking pilot product of wax oil, the mass difference of the collection bottle and the mass difference of the separation column, according to the following Equation 1) and Equation 2):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

in Equation 1) and Equation 2), W1 is the mass percentage (%) of asphaltenes in the sample to be measured;
W2 is the mass percentage (%) of benzene-based insoluble substances in the sample to be measured;
m₁ is the mass of the dried collection bottle used in step 6;
m₂ is the mass (g) of the dried separation column (filled with the filler) in step 1;
m₃ is the mass (g) of the sample to be measured;
m₄ is the mass (g) of the collection bottle after drying in step 7;
m₅ is the mass (g) of the separation column after drying in step 7.

In the example, at the stage of sample preparation, the mass of the catalytic cracking pilot product of wax oil, that is, m₃ was 100.5462 g; the mass of the collection bottle, that is, m₁ was 16.5342 g; the mass of the separation column, that is, m₂ was 12.4561 g; at the stage of measurement of insoluble substances, the mass of the collection bottle, that is, m₄ was 17.4894 g; and the mass of the separation column, that is, m₅ was 12.8382 g. Thus, in the catalytic cracking pilot product of wax oil, the mass of the n-pentane asphaltenes was 0.9552 g, and the mass of the toluene insoluble substances was 0.3821 g; the mass content of the n-pentane asphaltenes was 0.95%, and the mass content of the toluene insoluble substances was 0.38% in the catalytic cracking pilot product of wax oil.

The measurement of the above sample was repeated once, and the results showed that the mass content of the n-pentane asphaltenes was 0.98%, and the mass content of the toluene insoluble substances was 0.32% in the sample.

It can be seen that the insoluble substance separation process in the method provided by the example of the disclosure is simple and fast, and the whole separation process of the asphaltenes and the toluene insoluble substances takes only 20 min, which greatly improves the analysis speed. The reproducibility of the obtained mass contents of the n-pentane asphaltenes and the toluene insoluble substances is high with deviations of only 0.03% and 0.06%.

### Example 3

This example provides a method for simultaneously measuring the contents of n-hexane asphaltenes and toluene insoluble substances in a lubricating oil sample by solid phase extraction separation, which comprises the following specific steps.

### Step 1. Preparation

A clean sample injection tube, a collection bottle and a 6 mL separation column filled with a filler were dried at 130 °C for 2 h and then placed in a desiccator, and cooled to room temperature for use.

Herein, the filler used in the separation column was a mixture of 30% barite powder and 70% quartz sand, based on 100% of the total weight of the filler. The particle size of the filler was 20-40 mesh, and the filling height was 10% of the height of the separation column. Polyethylene having a pore size of 50 μm was used as both upper and lower filter materials.

Step 2. Sample Preparation 30.5463 g of the lubricating oil sample was diluted with n-hexane at a mass ratio of 1:5, ultrasonically oscillated at 50 °C and uniformly mixed, and the resulting mixture was placed in the sample injection tube.

The masses of the collection bottle and the separation column were recorded.

### Step 3. Activation

3 mL of n-hexane at 50 °C was added to the top of the separation column to activate the separation column.

### Step 4. Sample Injection

All the sample at 50 °C in the sample injection tube was added to the top of the separation column.

### Step 5. Washing

The sample injection tube was washed 5 times with 10 mL of n-hexane at 50 °C, and all the washing liquid was added to the top of the separation column, and the separation column was rinsed with the washing liquid.

### Step 6. Rinsing

The separation column was rinsed with 30 mL of n-hexane at 50 °C to obtain n-hexane insoluble substances.

A dried collection bottle was replaced, and the separation column was rinsed with 30 mL of toluene at 20 °C, whereby n-hexane asphaltenes entered the collection bottle, and toluene insoluble substances remained on the separation column.

### Step 7. Drying

The n-hexane in the collection bottle was evaporated. The evaporated collection bottle and the separation column were placed in an oven at 150 °C, dried for 1 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the n-hexane asphaltenes and the toluene insoluble substances was calculated from the mass of the lubricating oil sample, the mass difference of the collection bottle and the mass difference of the separation column, according to the following Equation 1) and Equation 2):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

in Equation 1) and Equation 2), W1 is the mass percentage (%) of asphaltenes in the sample to be measured;
W2 is the mass percentage (%) of benzene-based insoluble substances in the sample to be measured;
m₁ is the mass of the dried collection bottle used in step 6;
m₂ is the mass (g) of the dried separation column (filled with the filler) in step 1;
m₃ is the mass (g) of the sample to be measured;
m₄ is the mass (g) of the collection bottle after drying in step 7;
m₅ is the mass (g) of the separation column after drying in step 7.

In the example, at the stage of sample preparation, the mass of the lubricating oil sample, that is, m₃ was 30.5463 g; the mass of the collection bottle, that is, m₁ was 16.5384 g; the mass of the separation column, that is, m₂ was 5.1314 g; at the stage of measurement of insoluble substances, the mass of the collection bottle, that is, m₄ was 16.6697 g; and the mass of the separation column, that is, m₅ was 5.1650 g. Thus, in the lubricating oil sample, the mass of the n-hexane asphaltenes was 0.1313 g, and the mass of the toluene insoluble substances was 0.0336 g; the mass content of the n-hexane asphaltenes was 0.43%, and the mass content of the toluene insoluble substances was 0.11% in the lubricating oil sample.

The measurement of the above sample was repeated once, and the results showed that the mass content of the n-hexane asphaltenes was 0.40%, and the mass content of the toluene insoluble substances was 0.16% in the sample.

It can be seen that the insoluble substance separation process in the method provided by the example of the disclosure is simple and fast, and the whole separation process of the asphaltenes and the toluene insoluble substances takes only 25 min, which greatly improves the analysis speed. The reproducibility of the obtained mass contents of the n-hexane asphaltenes and the toluene insoluble substances is high with deviations of only 0.03% and 0.05%.

### Example 4

This example provides a method for simultaneously measuring the contents of petroleum ether asphaltenes and benzene insoluble substances in a coal tar sample by solid phase extraction separation, which comprises the following specific steps.

### Step 1. Preparation

A clean sample injection tube, a collection bottle and a 40 mL separation column filled with a filler were dried at 120 °C for 2 h and then placed in a desiccator, and cooled to room temperature for use.

Herein, the filler used in the separation column was a mixture of 20% calcium sulfate and 80% quartz sand, based on 100% of the total weight of the filler. The particle size of the filler was 200-300 mesh, and the filling height was 30% of the height of the separation column. Polyethylene having a pore size of 0.1 μm was used as the lower filter material, and a Teflon material having a pore size of 10 μm was used as the upper filter material.

### Step 2. Sample Preparation

2.8514 g of the coal tar sample was diluted with petroleum ether at a mass ratio of 1:30, ultrasonically oscillated at 40 °C and uniformly mixed, and the resulting mixture was placed in the sample injection tube.

The masses of the collection bottle and the separation column were recorded.

### Step 3. Activation

7 mL of petroleum ether at 40 °C was added to the top of the separation column to activate the separation column.

### Step 4. Sample Injection

All the sample at 40 °C in the sample injection tube was added to the top of the separation column.

### Step 5. Washing

The sample injection tube was washed twice with 15 mL of petroleum ether at 40 °C, and all the washing liquid was added to the top of the separation column, and the separation column was rinsed with the washing liquid.

### Step 6. Rinsing

The separation column was rinsed with 40 mL of petroleum ether at 40 °C to obtain petroleum ether insoluble substances.

A dried collection bottle was replaced, and the separation column was rinsed with 30 mL of benzene at 50 °C, whereby petroleum ether asphaltenes entered the collection bottle, and benzene insoluble substances remained on the separation column.

### Step 7. Drying

The petroleum ether in the collection bottle was evaporated. The evaporated collection bottle and the separation column were placed in an oven at 120 °C, dried for 1 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the petroleum ether asphaltenes and the benzene insoluble substances was calculated from the mass of the coal tar sample, the mass difference of the collection bottle and the mass difference of the separation column, according to the following Equation 1) and Equation 2):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

in Equation 1) and Equation 2), W1 is the mass percentage (%) of asphaltenes in the sample to be measured;
W2 is the mass percentage (%) of benzene-based insoluble substances in the sample to be measured;
m₁ is the mass of the dried collection bottle used in step 6;
m₂ is the mass (g) of the dried separation column (filled with the filler) in step 1;
m₃ is the mass (g) of the sample to be measured;
m₄ is the mass (g) of the collection bottle after drying in step 7;
m₅ is the mass (g) of the separation column after drying in step 7.

In the example, at the stage of sample preparation, the mass of the coal tar sample, that is, m₃ was 2.8514 g; the mass of the collection bottle, that is, m₁ was 16.6578 g; the mass of the separation column, that is, m₂ was 38.9568 g; at the stage of measurement of insoluble substances, the mass of the collection bottle, that is, m₄ was 16.8440 g; and the mass of the separation column, that is, m₅ was 38.9810 g. Thus, in the coal tar sample, the mass of the petroleum ether asphaltenes was 0.1862 g, and the mass of the benzene insoluble substances was 0.0242 g; the mass content of the petroleum ether asphaltenes was 6.53%, and the mass content of the benzene insoluble substances was 0.85% in the coal tar sample.

The measurement of the above sample was repeated once, and the results showed that the mass content of the petroleum ether asphaltenes was 6.49%, and the mass content of the benzene insoluble substances was 0.86% in the sample.

It can be seen that the insoluble substance separation process in the method provided by the example of the disclosure is simple and fast, and the whole separation process of the asphaltenes and the benzene insoluble substances takes only 30 min, which greatly improves the analysis speed. The reproducibility of the obtained mass contents of the petroleum ether asphaltenes and the benzene insoluble substances is high with deviations of only 0.04% and 0.01%.

### Example 5

This example provides a method for simultaneously measuring the contents of n-heptane asphaltenes and toluene insoluble substances in a coking slurry sample by solid phase extraction separation, which comprises the following specific steps.

### Step 1. Preparation

A clean sample injection tube, two identical collection bottles and a 30 mL separation column filled with a filler were dried at 150 °C for 1 h and then placed in a desiccator, and cooled to room temperature for use.

Herein, the filler used in the separation column was 100% quartz sand, based on 100% of the total weight of the filler. The particle size of the filler was 40-60 mesh, and the filling height was 50% of the height of the separation column. A filter cloth having a pore size of 10 μm was used as the lower filter material, and polyethylene having a pore size of 100 μm was used as the upper filter material.

### Step 2. Sample Preparation

6.8951 g of the coking slurry sample was diluted with n-pentane at a mass ratio of 1:10, ultrasonically oscillated at 50 °C and uniformly mixed, and the resulting mixture was placed in the sample injection tube.

The masses of the collection bottle and the separation column were recorded.

### Step 3. Activation

8 mL of n-pentane at 20 °C was added to the top of the separation column to activate the separation column.

### Step 4. Sample Injection

All the sample at 50 °C in the sample injection tube was added to the top of the separation column.

### Step 5. Washing

The sample injection tube was washed twice with 10 mL of n-pentane at 20 °C, and all the washing liquid was added to the top of the separation column, and the separation column was rinsed with the washing liquid.

### Step 6. Rinsing

The separation column was rinsed with 10 mL of n-pentane at 20 °C; the collection bottle under the separation column was replaced with a dried collection bottle (the first collection bottle), and the separation column was rinsed with 15 mL of n-heptane at 50 °C, whereby n-pentane insoluble and n-heptane soluble substances entered the collection bottle, and n-heptane insoluble substances remained in the separation column.

A dried collection bottle (the second collection bottle) was replaced, and the separation column was rinsed with 30 mL of toluene at 50 °C, whereby n-heptane asphaltenes entered the collection bottle, and toluene insoluble substances remained on the separation column.

### Step 7. Drying

The n-pentane and n-heptane in the collection bottle was evaporated. The evaporated collection bottle and the separation column were placed in an oven at 120 °C, dried for 1 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the n-pentane insoluble and n-heptane soluble substances, the n-heptane asphaltenes and the toluene insoluble substances was calculated from the mass of the coking slurry sample, the mass difference of the collection bottle and the mass difference of the separation column, according to the following Equation 1) to Equation 3):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

$$W 3 = ( {m}_{7} - {m}_{6} ) / {m}_{3} \times 100 \% ; \qquad Equation 3 )$$

in Equation 1) to Equation 3), W1 is the mass percentage (%) of asphaltenes in the sample to be measured;
W2 is the mass percentage (%) of benzene-based insoluble substances in the sample to be measured;
W3 is the mass percentage (%) of n-pentane insoluble and n-heptane soluble substances in the sample to be measured;
m₁ is the mass of the dried collection bottle (the second collection bottle) used in step 6;
m₂ is the mass (g) of the dried separation column (filled with the filler) in step 1;
m₃ is the mass (g) of the sample to be measured;
m₄ is the mass (g) of the collection bottle (the second collection bottle) after drying in step 7;
m₅ is the mass (g) of the separation column after drying in step 7.
m₆ is the mass of the dried collection bottle (the first collection bottle) used in step 6;
m₇ is the mass (g) of the collection bottle (the first collection bottle) after drying in step 7;

In the example, at the stage of sample preparation, the mass of the coking slurry sample, that is, m₃ was 6.8951 g; the masses of the collection bottles, that is, m₁ and m₆ were 16.5384 g and 16.4563 g, respectively; the mass of the separation column, that is, m₂ was 25.1268 g; at the stage of measurement of insoluble substances, the masses of the collection bottles, that is, m₄ and m₇ were 16.6094 g and 16.9948 g, respectively; and the mass of the separation column, that is, m₅ was 25.1875 g. Thus, in the coking slurry sample, the mass of the n-pentane insoluble and n-heptane soluble substances is 0.0710 g, the mass of the n-heptane asphaltenes was 0.5385 g, and the mass of the toluene insoluble substances was 0.0607 g; the mass content of the n-pentane insoluble and n-heptane soluble substances is 1.03%, the mass content of the n-heptane asphaltenes was 7.81%, and the mass content of the toluene insoluble substances was 0.88% in the coking slurry sample.

It can be seen that the insoluble substance separation process in the method provided by the example of the disclosure is simple and fast, and the mass contents of n-heptane asphaltenes, toluene insoluble substances and n-pentane insoluble and n-heptane soluble substances can be obtained simultaneously, which greatly improves the analysis speed.

### Comparative Example 1

In this comparative example, the content of n-heptane asphaltenes in the petrochemical asphalt sample of Example 1 was measured in accordance with the method of SH/T 0509-2010. Specifically, the procedure was as follows.

In a first ground joint conical flask that has been allowed to have a constant weight, 0.5 g of the sample was weighed and 25 mL of n-heptane was added. The first ground joint conical flask containing the sample and n-heptane was connected to a condenser and heated for recovery over 1 h. The reflux rate of the condensing solvent was controlled to be dropwise rather than linear. When the solution was cooled, the first ground joint flask was removed and covered with a stopper, and left to stand and settle in a dark place for 1 h.

Without shaking, the supernatant was slowly poured as much as possible into a funnel with a quantitative filter paper, and finally the remaining small amount of solution and precipitate were shaken and poured onto the filter paper, with taking care not to raise the solution to the upper edge of the filter paper. The residue in the first ground joint conical flask was washed several times with 30 mL of hot n-heptane at 60-70 °C, and the washing liquid was also poured onto the filter paper. All the filtrate was collected in a second ground joint conical flask, and the first ground joint conical flask did not need to be washed. The filter paper with the precipitate was folded and placed into an extractor. The second ground joint conical flask was assembled with the extractor and a condenser, and heated to reflux for 1 h or until the dripping liquid was colorless. Then the reflux was complete and it was cooled.

60 mL of toluene was added to the first ground joint conical flask, which was assembled with an extractor and a condenser, and refluxed for 1 h or until the dripping liquid was colorless. After cooling, the first ground joint conical flask was removed and toluene was evaporated. Then it was placed into a vacuum oven, and kept at a temperature of 105 °C and a vacuum degree of 93 kPa for 1 h. When it was taken out, it was cooled to room temperature in a desiccator filled with a desiccant, and the mass of the obtained asphaltenes was weighed. The whole separation process took 6 h.

The petrochemical asphalt sample was measured twice to obtain the content of n-heptane asphaltenes, which was 20.8% and 19.9%, respectively. It can be seen that the determination of n-heptane asphaltenes in the petrochemical asphalt sample by SH/T 0509-2010 has complicated steps, is time-consuming, uses a large amount of solvent, and is poor in reproducibility, and the content of toluene insoluble substance cannot be determined at the same time.

### Comparative Example 2

In this comparative example, the toluene insoluble substance in the petrochemical asphalt sample of Example 1 was separated in accordance with GB/T 2292-2018 “Determination of toluene insoluble content for coking products”. Specifically, the procedure was as follows.

10 g of treated quartz sand was first poured into a filter paper cylinder, placed in a weighing bottle, and dried in a drying oven at 120 °C to a constant weight. Subsequently, 1 g of the sample was weighed, and the sample and the quartz sand were fully stirred and uniformly mixed in the filter paper cylinder.

A flat-bottomed flask containing 120 mL of toluene was placed in an electric heating mantle. The filter paper cylinder was placed in an extraction cylinder, such that the upper edge of the filter paper cylinder was 20 mm higher than the reflux tube, and the extraction cylinder was connected to the flat-bottomed flask. After that, about 30 mL of toluene was added along the inner wall of the filter paper cylinder. A condenser hung with drainage iron wire was connected to the extraction cylinder, and cooling water was connected. At the same time, the photoelectric probe of the intelligent counter was horizontally clamped on the reflux tube, and extraction was carried out 60 times. The electric heating mantle was turned on to heat the flat-bottom flask, and the speed of toluene extraction was controlled at 1 min/time to 1.5 min/time. The toluene extract fully flowing back to the flat-bottom flask from the reflux tube is considered as one extraction. When the filter paper cylinder was cleaned by the toluene extract, such that the filter paper cylinder was white or pale yellow and the extract in the extraction cylinder was clarified, this was the end point of extraction. At this time, the heating was stopped, and the power supply of the electric heating mantle was disconnected. The whole extraction process took 180 min.

After the heating was stopped and it was slightly cooled, the filter paper cylinder was removed and placed in the original weighing bottle without capping, which was then placed in a ventilation cabinet. When toluene was evaporated, the weighing bottle with the cap was placed in a drying oven at 120 °C for 2 h. After that, the weighing bottle was taken out from the drying oven, immediately capped, placed in a desiccator, cooled to room temperature for weighing, and dried for 0.5 h before constant weight checking, until the mass difference of the dried filter paper cylinder obtained in 2 consecutive times did not exceed 0.001 g or the mass did not increase any more. The last mass was taken as the basis for calculation; if there was an increase in weight, the mass before the increase was taken as the basis for calculation.

The petrochemical asphalt sample was measured twice to obtain the content of toluene insoluble substance, which was 10.8% and 11.6%, respectively. It can be seen that the determination of toluene insoluble substances in the petrochemical asphalt sample by GB/T 2292-2018 has complicated steps, is time-consuming and poor in reproducibility, and the content of n-heptane asphaltenes cannot be determined at the same time.

### Comparative Example 3

In Comparative Example 3, the contents of n-pentane asphaltenes and toluene insoluble substances in the catalytic cracking pilot product of wax oil in Example 2 were measured in accordance with SH/T 0509-2010 and GB/T 2292-2018. Specifically, the procedure was as follows.

A slow quantitative filter paper cylinder was soaked in toluene for 24 h and then dried, and two layers were folded concentrically and placed into a clean weighing bottle while ensuring that it can be completely sealed with a cover. The total mass of the weighing flask and the filter paper cylinder was weighed, and the mass of a 150 mL ground joint conical flask was weighed at the same time.

2.0 g of the sample to be measured was weighed and placed in the filter paper cylinder, and then the filter paper cylinder with the sample was placed in an extractor and a certain amount of n-pentane was added for reflux. Heating was stopped until the filtrate in the extractor was nearly colorless. When it was cooled to room temperature and the n-pentane filtrate in the filter paper cylinder was completely filtered, the n-pentane soluble substances were discarded; the conical flask that had been allowed to have a constant weight was replaced, and a certain amount of toluene was added into the extractor for reflux. During this experiment, the amount of residual n-pentane contained in the filter paper cylinder was very small, and the n-pentane soluble substances in the sample had been completely filtered, so the residual n-pentane after the addition of toluene did not affect the measurement results.

When the toluene filtrate in the extractor was nearly colorless, heating was stopped. When cooled to room temperature, the filter paper cylinder was taken out and placed into a corresponding weighing bottle. After toluene in the filter paper cylinder was completely evaporated, the total mass of the weighing bottle and the filter paper cylinder was weighed. The 150 mL ground joint conical flask was heated in an electric heating mantle until toluene in the flask was evaporated to near dryness, and the flask was placed in a vacuum drying oven for 1 h and then weighed. The contents of toluene insoluble substances and n-pentane asphaltenes were calculated according to the relevant equations in the standards.

The catalytic cracking pilot product of wax oil was measured twice to obtain the content of toluene insoluble substances and the content of n-pentane asphaltenes. The content of n-pentane asphaltenes was 0.98% and 0.79% respectively, and the content of toluene insoluble substances was 0.35% and 0.23% respectively, with a deviation of 0.19% and 0.12% respectively.

Compared with the experimental results in Example 2, it can be seen that the mass content of n-pentane asphaltenes and the mass content of toluene insoluble substances obtained in this comparative example have a large deviation and low reproducibility, and the steps are complicated and time-consuming.

### Comparative Example 4

In this comparative example, the content of benzene insoluble substances in the coal tar sample of Example 4 was measured in accordance with GB/T 2559.7-2005 “Analysis of lignite wax-Method for measuring benzene insolubles in lignite wax”. Specifically, the procedure was as follows.

The filter paper cylinder was placed into a high weighing bottle. With the cap opened, the weighing bottle was placed into a drying oven which had been blown and heated to 102 °C, and dried for 1 h. Subsequently, the weighing bottle was taken out, immediately capped, placed into a desiccator, cooled for 30 min and then weighed. Inspection drying was performed for 30 min each time until the mass change did not exceed 0.0005 g after two consecutive times of drying.

5 g of the sample with a particle size of less than 1 mm was weighed and placed into the filter paper cylinder that had been allowed to have a constant mass. 100 mL of benzene was added to the ground joint conical flask of the extraction device, and a reflux device was installed, where the filter paper cylinder could not contact the benzene liquid phase in the ground joint conical flask. Extraction was carried out in a water bath at 90 °C for 3 h until colorless benzene liquid was dripped from the filter paper cylinder.

After the residual benzene in the filter paper cylinder was drained off, the filter paper cylinder was taken out and placed into the original weighing bottle. With the cap opened, the weighing bottle was first pre-dried under an infrared lamp, and then it was placed into a drying oven which had been heated to 102 °C, and dried for 1 h. Subsequently, the weighing bottle was taken out, immediately capped, placed into a desiccator, cooled for 30 min and then weighed. Inspection drying was performed for 30 min each time until the mass change did not exceed 0.0005 g after two consecutive times of drying.

The coal tar sample was measured twice to obtain the content of benzene insoluble substances, which was 0.61% and 0.85% respectively. It can be seen that the traditional analysis method of benzene insoluble substances is time-consuming, complicated, and poor in reproducibility of the obtained data.

### Comparative Example 5

In this comparative example, the filler used in the separation column was changed, and the contents of n-heptane asphaltenes and toluene insoluble substances in the coking slurry sample of Example 5 was measured by solid phase extraction separation provided in Example 5. Specifically, the procedure was as follows.

### Step 1. Preparation

A clean sample injection tube, a collection bottle and a 30 mL separation column filled with a filler were dried at 150 °C for 1 h and then placed in a desiccator, and cooled to room temperature for use.

Herein, the filler used in the separation column was 100% silica gel. The particle size of the filler was 40-60 mesh, and the filling height was 50% of the height of the separation column. A filter cloth having a pore size of 10 μm was used as the lower filter material, and polyethylene having a pore size of 100 μm was used as the upper filter material.

### Step 2. Sample Preparation

6.3452 g of the coking slurry sample was diluted with n-pentane at a mass ratio of 1:10, ultrasonically oscillated at 50 °C and uniformly mixed, and the resulting mixture was placed in the sample injection tube.

The masses of the collection bottle and the separation column were recorded.

### Step 3. Activation

8 mL of n-pentane at 20 °C was added to the top of the separation column to activate the separation column.

### Step 4. Sample Injection

All the sample at 50 °C in the sample injection tube was added to the top of the separation column.

### Step 5. Washing

The sample injection tube was washed twice with 10 mL of n-pentane at 20 °C, and all the washing liquid was added to the top of the separation column, and the separation column was rinsed with the washing liquid.

### Step 6. Rinsing

The separation column was rinsed with 20 mL of n-pentane at 20 °C; the collection bottle under the separation column was replaced with a dried collection bottle (the first collection bottle), and the separation column was rinsed with 30 mL of n-heptane at 50 °C, whereby n-pentane insoluble and n-heptane soluble substances entered the collection bottle, and n-heptane insoluble substances remained in the separation column.

A dried collection bottle (the second collection bottle) was replaced, and the separation column was rinsed with 100 mL of toluene at 50 °C, whereby n-heptane asphaltenes entered the collection bottle, and toluene insoluble substances remained on the separation column.

### Step 7. Drying

The n-pentane and n-heptane in the collection bottle was evaporated. The evaporated collection bottle and the separation column were placed in an oven at 120 °C, dried for 1 h and cooled to room temperature in a desiccator, and the masses of the collection bottle and the separation column were weighed. Subsequently, the collection bottle and the separation column were dried for 0.5 h and cooled to room temperature, and the masses of the collection bottle and the separation column were weighed again. If the mass differences of the collection bottle and the separation column obtained by weighing twice were not greater than 0.001 g, the drying was stopped; otherwise, the collection bottle and the separation column were dried again until the mass differences of the collection bottle and the separation column after drying twice were less than 0.001 g.

### Step 8. Calculation

The content of each of the n-pentane insoluble and n-heptane soluble substances, the n-heptane asphaltenes and the toluene insoluble substances was calculated from the mass of the coking slurry sample, the mass difference of the collection bottle and the mass difference of the separation column, according to the following Equation 1) to Equation 3):

$$W 1 = ( {m}_{4} - {m}_{1} ) / {m}_{3} \times 100 \% ; \qquad Equation 1 )$$

$$W 2 = ( {m}_{5} - {m}_{2} ) / {m}_{3} \times 100 \% ; \qquad Equation 2 )$$

$$W 3 = ( {m}_{7} - {m}_{6} ) / {m}_{3} \times 100 \% ; \qquad Equation 3 )$$

in Equation 1) to Equation 3), W1 is the mass percentage (%) of asphaltenes in the sample to be measured;
W2 is the mass percentage (%) of benzene-based insoluble substances in the sample to be measured;
W3 is the mass percentage (%) of n-pentane insoluble and n-heptane soluble substances in the sample to be measured;
m₁ is the mass of the dried collection bottle (the second collection bottle) used in step 6;
m₂ is the mass (g) of the dried separation column (filled with the filler) in step 1;
m₃ is the mass (g) of the sample to be measured;
m₄ is the mass (g) of the collection bottle (the second collection bottle) after drying in step 7;
m₅ is the mass (g) of the separation column after drying in step 7.
m₆ is the mass of the dried collection bottle (the first collection bottle) used in step 6;
m₇ is the mass (g) of the collection bottle (the first collection bottle) after drying in step 7;

In the example, at the stage of sample preparation, the mass of the coking slurry sample, that is, m₃ was 6.3452 g; the masses of the collection bottles, that is, m₁ and m₆ were 16.3568 g and 16.4561 g; the mass of the separation column, that is, m₂ was 24.9123 g; at the stage of measurement of insoluble substances, the masses of the collection bottles, that is, m₄ and m₇ were 16.4767 g and 16.8984 g, respectively; and the mass of the separation column, that is, m₅ was 25.1038 g. Thus, in the coking slurry sample, the mass of the n-pentane insoluble and n-heptane soluble substances is 0.1199 g, the mass of the n-heptane asphaltenes was 0.4423 g, and the mass of the toluene insoluble substances was 0.1935 g; the mass content of the n-pentane insoluble and n-heptane soluble substances is 1.89%, the mass content of the n-heptane asphaltenes was 6.97%, and the mass content of the toluene insoluble substances was 3.05% in the coking slurry sample.

Compared with Example 5, it can be seen that when the contents of n-heptane asphaltenes and toluene insoluble substances in the coking slurry sample of Example 5 were measured simultaneously with the method in Comparative Example 5, the amount of solvent used is large and it is time-consuming; and compounds such as strongly polar aromatics remain in the extraction column, so that the content of toluene insoluble substances is high, and the accuracy of the analysis result is poor.

In summary, the method for simultaneously measuring the contents of asphaltenes and benzene-based insoluble substances by solid phase extraction separation provided in Examples of the disclosure can be applied to the determination of asphaltenes and benzene-based insoluble substances in samples of coal chemical industry, petrochemical industry, or the like. The method is simple and reliable, has high reproducibility, high analysis efficiency and low solvent consumption, and can realize the automation of the analysis process by means of instruments. It solves the problems (such as instrument error introduced by the separation consumables, low extraction efficiency/low separation efficiency, complicated steps, long separation time/long time consumption, and large solvent consumption) of the traditional methods for separating insoluble substances based on liquid-liquid extraction or centrifugal method, and it has high promotion value and application potential. In addition, the method of the disclosure can also be implemented separately to separate alkane insoluble substances and benzene-based insoluble substances in samples of coal chemical industry, petrochemical industry, or the like.

The above description is only specific examples of the disclosure, and cannot limit the implementable scope of the disclosure, so the replacement with its equivalent component, or the equivalent changes and modifications made according to the protection scope of the present patent for invention, should still fall within the scope of this patent. In addition, a technical feature and another technical feature, a technical feature and a technical invention, and a technical invention and another technical invention in the disclosure can be used in any arbitrary combination.

1. A method for simultaneously measuring the contents of asphaltenes and benzene-based insoluble substances, comprising:
(1) mixing a sample to be measured with a diluent under heating conditions until uniform and placing the obtained mixture in a sample injection tube;
(2) activating a dried separation column with the diluent;
(3) adding all the mixture in the sample injection tube to the top of the separation column;
(4) rinsing the separation column with the diluent to obtain alkane insoluble substances; then rinsing the separation column with a benzene-based eluent, and collecting an effluent with a dried collection bottle and stopping rinsing until the effluent is colorless, whereby asphaltenes are in the collection bottle, and benzene-based insoluble substances remain on the separation column;
(5) drying the collection bottle and the separation column in (4); and
(6) calculating the content of each of the asphaltenes and the benzene-based insoluble substances according to the mass of the sample to be measured, the mass difference of the collection bottle, and the mass difference of the separation column.

2. The method according to claim 1, further comprising, between (3) and (4), washing the sample injection tube with the diluent and adding all the resultant washing liquid to the top of the separation column;
wherein the amount of the diluent used for washing the sample injection tube is 5-50 mL, and the temperature of the diluent used for washing the sample injection tube is 20-100 °C.

3. The method according to claim 1, wherein the diluent comprises one or more of n-pentane, n-hexane, n-heptane and petroleum ether.

4. The method according to claim 1, wherein the sample to be measured comprises crude oil, heavy oil, tar or asphalt products in coal chemical industry or petrochemical industry.

5. The method according to claim 1, wherein the amount of the sample to be measured is 0.1-100 g.

6. The method according to claim 1, wherein the volume of the separation column is 6-150 mL.

7. (canceled)

8. The method according to claim 1, wherein a filter material used in the separation column has a pore size of 0.1-200 μm.

9. The method according to claim 8, wherein the filter material comprises a neutral filter paper, a filter cloth, a glass sand core, or a polymeric material.

10. (canceled)

11. The method according to claim 1, wherein the filling height of a filler used in the separation column is 5%-80% of the total height of the separation column, and the particle size of the filler is in the range of 20-300 mesh.

12. The method according to claim 1, wherein a filler used in the separation column is a weakly polar material and/or an inert material.

13. The method according to claim 12, wherein the filler is quartz sand or a mixture of quartz sand and at least one selected from the group consisting of calcium sulfate, alumina, silica gel, diatomaceous earth, clay, polymer, and barite powder.

14. The method according to claim 13, wherein the content of quartz sand in the filler is 70%-100%, based on 100% of the total weight of the filler.

15. The method according to claim 1, wherein in (1), the mass ratio of the sample to be measured to the diluent is 1:1 to 1:50;
in (2), the amount of the diluent is 3-20 mL.

16. (canceled)

17. The method according to claim 1, wherein in (4), the amount of the diluent is 1-10 times the amount of the diluent in (2), and the amount of the benzene-based eluent is 1-10 times the amount of the diluent in (2).

18. The method according to claim 1, wherein the benzene-based eluent comprises benzene and/or toluene.

19. The method according to claim 1, wherein in (2), (4) and (5), the temperature for drying is 100-150 °C.

20. The method according to claim 1, wherein in (1), the temperature for heating is 50-100 °C.

21. The method according to claim 1, wherein in (2), the temperature of the diluent is 20-100 °C.

22. The method according to claim 1, wherein in (4), the temperature of the diluent and the benzene-based eluent is 20-100 °C.

23. (canceled)

24. The method according to claim 1, wherein in (6), the content of each of the asphaltenes and the benzene-based insoluble substances in the sample to be measured is calculated from the mass of the sample to be measured, the mass difference of the collection bottle and the mass difference of the separation column, according to the following Equation 1) and Equation 2):
W1=(m₄−m₁)/m₃×100% Equation 1);
W2−(m₅−m₂)/m₃×100% Equation 2);
in Equation 1) and Equation 2), W1 is the mass percentage (%) of the asphaltenes in the sample to be measured;
W2 is the mass percentage (%) of the benzene-based insoluble substances in the sample to be measured;
m₁ is the mass (g) of the dried collection bottle used in (4);
m₂ is the mass (g) of the dried separation column in (2);
m₃ is the mass (g) of the sample to be measured;
m₄ is the mass (g) of the collection bottle after drying in (5); and
m₅ is the mass (g) of the separation column after drying in (5).
